(19) United States
(12) Patent Application Publication
(10) Pub. No.: US 2023/0248324 A1
(43) Pub. Date: Aug. 10, 2023

(54) X-RAY DIAGNOSTIC APPARATUS AND MEDICAL COUCH APPARATUS

(71) Applicant: CANON MEDICAL SYSTEMS CORPORATION, Otawara-shi (JP)

(72) Inventors: Toshikatsu OOHASHI, Otawara (JP); Suzuna SAITO, Sakura (JP); Motohiro SATO, Nasushiobara (JP); Yoshinori SHIMIZU, Nasushiobara (JP); Yusuke OKUMURA, Nasushiobara (JP); Naoko KURATOMI, Sakura (JP); Yoshinori SAITO, Otawara (JP)

(73) Assignee: CANON MEDICAL SYSTEMS CORPORATION, Otawara-shi (JP)

(21) Appl. No.: 18/162,781

(22) Filed: Feb. 1, 2023

(30) Foreign Application Priority Data
Feb. 9, 2022 (JP) 2022-018927

(51) Int. Cl. A61B 6/04; A61B 6/00

(52) U.S. Cl. CPC A61B 6/0407; A61B 6/4452; A61B 6/4482

(57) ABSTRACT

An X-ray diagnostic apparatus according to an embodiment includes a couchtop, an irradiation unit, a support member, a first opening, and an X-ray detector. The couchtop is a component on which a subject is placed. The irradiation unit outputs X-rays from above the subject. The support member supports both ends of the couchtop in the lateral direction from below and forms a gap between the support member and the couchtop in a center part corresponding to an area excluding both ends. The first opening is formed at either one or both of the ends of the couchtop in the longitudinal direction and is accessible to the gap. The X-ray detector is provided in the gap through the first opening and detects the X-rays.

### CROSS-REFERENCE TO RELATED APPLICATIONS

This application is based upon and claims the benefit of priority from Japanese Patent Application No. 2022-018927, filed on Feb. 9, 2022, the entire contents of which are incorporated herein by reference.

### FIELD

Embodiments described herein relate generally to an X-ray diagnostic apparatus and a medical couch apparatus.

### BACKGROUND

Conventionally, typical X-ray imaging apparatuses have been using a couchtop provided at a position sandwiched between an X-ray tube and an X-ray detector to perform imaging on a subject placed on the couchtop.

### BRIEF DESCRIPTION OF THE DRAWINGS

FIG. 1 is a block diagram of an example of the configuration of an X-ray diagnostic apparatus according to an embodiment;

FIG. 2 is a perspective view of an example of the configuration of a couchtop of the X-ray diagnostic apparatus according to the embodiment;

FIG. 3 is a perspective view of an example of the configuration of an end of the couchtop of the X-ray diagnostic apparatus according to the embodiment;

FIG. 4 is a view of an example of a urological examination using the X-ray diagnostic apparatus according to the embodiment;

FIG. 5 is a front view of the end of the couchtop in the longitudinal direction according to the embodiment;

FIG. 6 is a view for explaining an example of insertion and removal of an X-ray detector according to the embodiment;

FIG. 7 is a view of an example of an opening when a shutter is closed according to the embodiment;

FIG. 8 is a perspective view of an example of the configuration of the X-ray diagnostic apparatus according to a first modification;

FIG. 9 is a perspective view of an example of the configuration of the X-ray diagnostic apparatus according to a second modification; and

FIG. 10 is a front view of the end of the couchtop in the longitudinal direction according to a third modification.

### DETAILED DESCRIPTION

An X-ray diagnostic apparatus according to an embodiment includes a couchtop, an irradiation unit, a support member, a first opening, and an X-ray detector. The couchtop is a component on which a subject is placed. The irradiation unit outputs X-rays from above the subject. The support member supports both ends of the couchtop in the lateral direction from below and forms a gap between the support member and the couchtop in a center part corresponding to an area excluding both ends. The first opening is formed at either one or both of the ends of the couchtop in the longitudinal direction and is accessible to the gap. The X-ray detector is provided in the gap through the first opening and detects the X-rays.

An X-ray diagnostic apparatus and a medical couch apparatus according to the present embodiment are described below with reference to the accompanying drawings. The following describes an example where the X-ray diagnostic apparatus according to the present embodiment is a typical X-ray imaging apparatus. In the following embodiments, parts denoted by like reference numerals are assumed to perform the same operations, and overlapping explanation thereof is appropriately omitted.

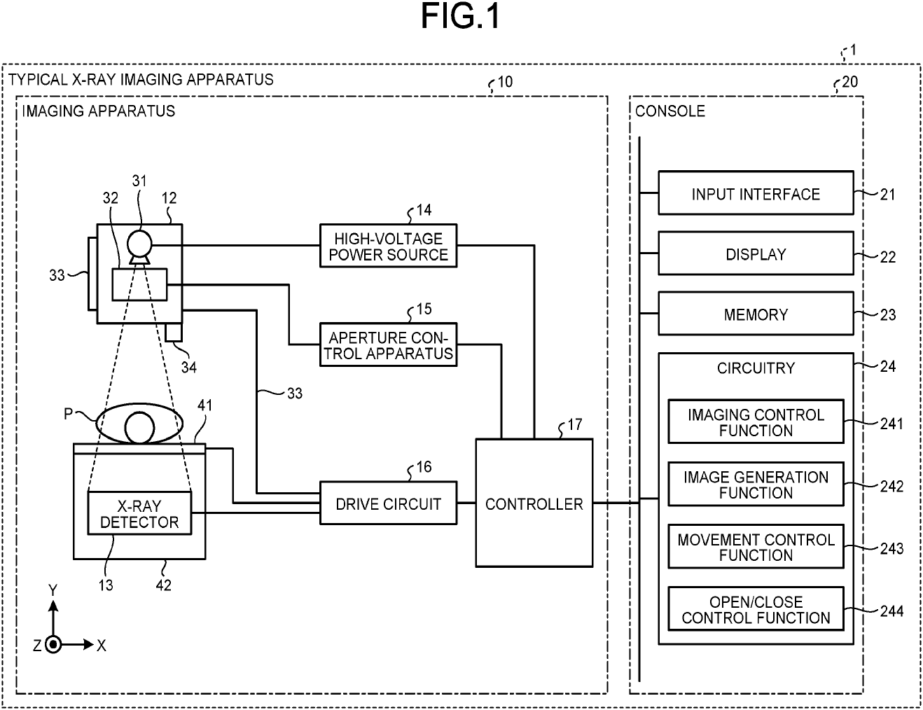

FIG. 1 is a block diagram of an example of the configuration of the X-ray diagnostic apparatus according to the present embodiment. As illustrated in FIG. 1, a typical X-ray imaging apparatus 1 includes an imaging apparatus 10 and a console 20. The imaging apparatus 10 is an example of an imaging unit.

The imaging apparatus 10 includes an X-ray tube holding apparatus 12, an X-ray detector 13, a high-voltage power source 14, an aperture control apparatus 15, a drive circuit 16, a controller 17, and a couch apparatus 42. For example, to perform X-ray imaging on a subject P lying horizontally, the subject P lying horizontally is placed on a couchtop 41 of the couch apparatus 42 as illustrated in FIG. 1. The imaging apparatus 10 may include a separate stand for imaging a standing subject P.

The X-ray tube holding apparatus 12 includes an X-ray tube 31, an aperture unit 32, an operation panel 33, and a camera 34.

The X-ray tube 31 is a vacuum tube that outputs thermal electrons from a cathode (filament) to an anode (target) by the application of high voltage from the high-voltage power source 14. The X-ray tube 31 is an example of an irradiation unit. The X-ray tube 31 is disposed facing the X-ray detector 13 across the subject P.

The aperture unit 32 includes a plurality of blades made of metal, such as lead, that blocks X-rays. The aperture unit 32 has a mechanism that adjusts the irradiation field of X-rays generated by the X-ray tube 31.

The operation panel 33 is provided to a housing of the X-ray tube holding apparatus 12. The operation panel 33 includes hard keys, such as buttons, and a display input apparatus that supply, when pressed by a user, unique instruction signals to a processor. The display input apparatus includes a display serving as a display unit and a touch sensor serving as an input unit provided near the display.

The display of the operation panel 33 displays various images, such as images indicating information on the typical X-ray imaging apparatus 1 and reference images of the subject P taken by the camera 34. The user can input various instructions for the image displayed on the display to the typical X-ray imaging apparatus 1 using the touch sensor and the hard keys of the operation panel 33. The operation panel 33 outputs signals corresponding to the input from the user to circuitry 24 of the console 20.

The camera 34 is provided to the housing of the X-ray tube holding apparatus 12. The camera 34 is composed of a charge coupled device (CCD) image sensor or a complementary metal oxide semiconductor (CMOS) image sensor. The camera 34 captures an image of the subject P standing on a stand 11 or placed horizontally on the couchtop 41 and outputs the captured image to the circuitry 24 of the console 20 via the controller 17. The camera 34 may be provided with a wide-angle or fisheye lens such that it can acquire a camera image of the subject P in a wider range. The camera 34 may be provided to a wall surface, including a ceiling of an examination room.

The X-ray detector 13 is composed of a flat panel detector (FPD) including a plurality of X-ray detection elements arrayed two-dimensionally. The X-ray detector 13 detects X-rays passing through the subject P and traveling to the X-ray detector 13 and outputs X-ray projection data based on the detected X-rays. The projection data is supplied to the console 20 via the controller 17. The X-ray detector 13 may include an image intensifier, a TV camera, or the like. The X-ray detector 13 is hereafter also referred to as an FPD 13.

The high-voltage power source 14 is composed of a high-voltage generation apparatus and an X-ray control apparatus. The high-voltage generation apparatus is composed of electrical circuits, such as a transformer and a rectifier, and has the function of generating high voltage to be applied to the X-ray tube 31. The X-ray control apparatus controls output voltage depending on the X-rays output by the X-ray tube 31.

The aperture control apparatus 15 controls the aperture unit 32. For example, the aperture control apparatus 15 controls the aperture unit 32 via the controller 17 to adjust the irradiation range of X-rays output from the X-ray tube 31.

The drive circuit 16 controls the drive of each unit of the typical X-ray imaging apparatus 1. For example, the drive circuit 16 controls the X-ray tube 31 via the controller 17 to move the X-ray tube 31 in conjunction with the movement of the X-ray detector 13 so as to maintain the state where the position of the center of the X-ray tube 31 faces the position of approximately the center of the X-ray detector 13.

Alternatively, for example, the drive circuit 16 controls the X-ray tube holding apparatus 12 and the X-ray detector 13 via the controller 17 to move them in conjunction with each other so as to maintain the positional relation between the position of the center of the X-ray tube 31 and the X-ray detector 13.

The controller 17 includes at least a processor and a memory. The controller 17 is controlled by the console 20 according to a computer program stored in the memory to collectively control each component of the imaging apparatus 10. For example, the controller 17 controls an X-ray irradiation system, thereby performing long imaging for generating a long image of the subject P. Thus, the controller 17 generates projection data and outputs it to the console 20.

The couch apparatus 42 includes the couchtop 41 on which the subject P is placed. The drive circuit 16 controls the couchtop 41 via the controller 17 to move it in the thickness direction (Y-axis direction) of the couchtop 41. Similarly, the drive circuit 16 controls the couchtop 41 to move it in the long axis direction (Z-axis direction).

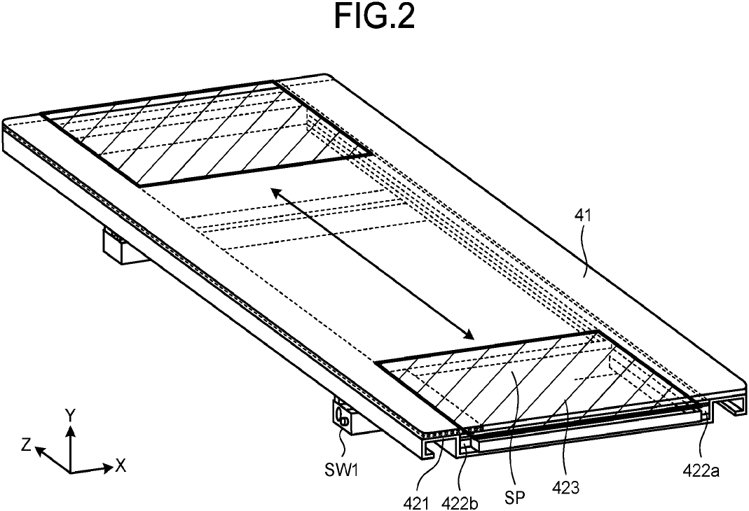

The configuration of the couch apparatus 42 is described with reference to FIGS. 2 to 7. FIG. 2 is a perspective view of an example of the configuration of the couchtop of the X-ray diagnostic apparatus according to the embodiment. As illustrated in FIG. 2, the couch apparatus 42 includes a reinforcement frame 421, guide rails 422a and 422b, and an FPD tray 423 besides the couchtop 41.

The reinforcement frame 421 is a member that supports the couchtop 41. The reinforcement frame 421 is an example of a support member. The reinforcement frame 421 is provided to a base (not illustrated) that supports the couchtop 41 and the reinforcement frame 421. The reinforcement frame 421 extends in the longitudinal direction of the couchtop 41 and supports the bottom surface of the couchtop 41 at both ends in the lateral direction (X-axis direction).

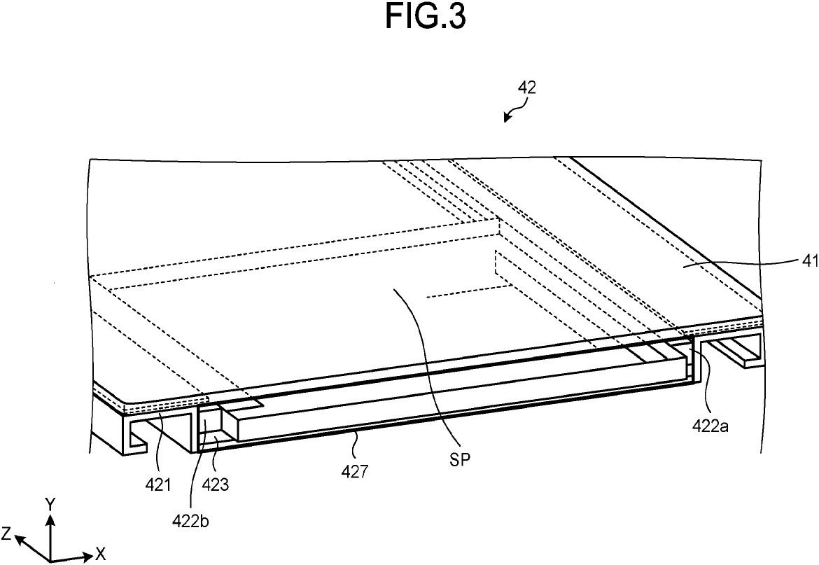

FIG. 3 is a perspective view of an example of the configuration of an end of the couchtop of the X-ray diagnostic apparatus according to the embodiment. As illustrated in FIG. 3, the reinforcement frame 421 has recesses having a recessed shape in the lateral section and forms a space SP (gap) between the couchtop 41 and the reinforcement frame 421 in a center part in the lateral direction excluding both ends described above. The center part corresponds to the area on which the subject P is placed. The end of the space SP in the longitudinal direction is open to form an opening 427. The opening 427 may be formed at one end or both ends in the longitudinal direction.

The opening 427 has such a size that the FPD 13 can be inserted into and removed from it, and the FPD 13 can be placed in the space SP through the opening 427. Specifically, the space SP is provided with the FPD tray 423 into and from which the FPD 13 can be inserted and removed. The user can place the FPD 13 in the space SP by inserting and removing the FPD 13 into and from the FPD tray 423 using the opening 427. The FPD tray 423 is provided movably in the longitudinal direction of the couchtop 41. The opening 427 will be described later.

While the FPD 13 according to the present embodiment can be inserted into and removed from the FPD tray 423, the FPD 13 may be fixed in the space SP and unable to be inserted into or removed from the FPD tray 423 by, for example, integrating the FPD 13 and the FPD tray 423.

The guide rails 422a and 422b are provided in the space SP formed between the couchtop 41 and the reinforcement frame 421. The guide rails 422a and 422b are provided extending in the longitudinal direction in parallel with each other at both ends of the reinforcement frame 421 in the lateral direction. The guide rails 422a and 422b support the FPD tray 423 with sliding members, such as sliders, interposed therebetween, thereby supporting the FPD tray 423 slidably in the longitudinal direction of the reinforcement frame 421.

The FPD tray 423 houses the FPD 13. The FPD tray 423 is an example of a housing unit. The FPD tray 423 is positioned at the end in the space SP connectable to the opening 427 and insertably and removably supports the FPD 13.

The FPD tray 423 is attached to the guide rails 422a and 422b with the sliding members, such as sliders, interposed therebetween. The sliders slide on the guide rails 422a and 422b by the drive of a drive source, such as a motor and an actuator. The guide rails 422a and 422b, the sliders, and the drive source, such as a motor and an actuator, are examples of a first movement mechanism.

The following describes the movement of the FPD tray 423 with reference to FIG. 2. FIGS. 2 and 3 illustrate the FPD tray 423 in which the FPD 13 is not housed. As illustrated in FIG. 3, the couch apparatus 42 includes a drive switch SW1.

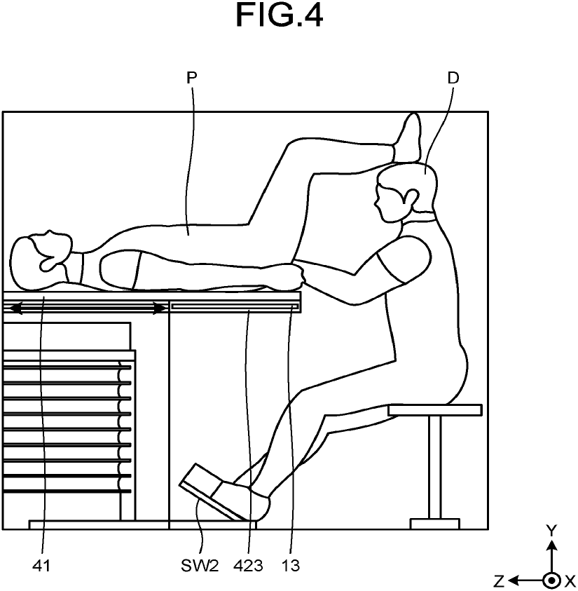

The drive switch SW1 is a switch for driving the drive source that slides the FPD tray 423. By operating the drive switch SW1, the user moves the FPD tray 423 and adjusts the position of the FPD tray 423. The couchtop 41 may also be moved by the drive switch SW1. The FPD tray 423 may be moved by a foot switch SW2 (refer to FIG. 4) or other switches.

The FPD tray 423 slides on the guide rails 422a and 422b and moves from one end to the other of the couchtop 41 in the longitudinal direction (refer to the arrow in FIG. 2) by the user’s operation. As a result, the FPD 13 housed in the FPD tray 423 can move between one end and the other of the couchtop 41. The X-ray tube 31 moves to follow the position of the FPD tray 423 (FPD 13) so as to maintain the positional relation between the X-ray tube 31 and the FPD 13 under the control of a movement control function 243 of the circuitry 24, which will be described later. Thus, X-ray imaging can be performed on the subject P at the end of the couchtop 41.

Therefore, the typical X-ray imaging apparatus 1 according to the present embodiment can perform X-ray imaging on a region of interest of the subject P if the region of interest is positioned at one end of the couchtop 41. For example, in X-ray imaging for a urological examination, the user may perform X-ray imaging on the subject lying supine on the couchtop with his or her legs open while inserting a catheter into the urethra. In such a case, positioning the lower back of the subject at the end of the couchtop facilitates the user’s inserting the catheter. The typical X-ray imaging apparatus 1 according to the present embodiment can perform imaging at the end position, thereby improving the convenience in urological examinations.

FIG. 4 is a view of an example of a urological examination using the X-ray diagnostic apparatus according to the embodiment. In the example illustrated in FIG. 4, an examiner D (user), such as a doctor, adjusts the positions of the couchtop 41 and the FPD tray 423 by operating the foot switch SW2.

As described above, the FPD 13 is housed in the FPD tray 423 and can be moved to the end of the couchtop 41 by the FPD tray 423 sliding on the guide rails 422a and 422b. The X-ray tube 31 also moves to follow the position of the FPD tray 423 so as to maintain the positional relation between the position of the center of the X-ray tube 31 and the FPD tray 423 (FPD 13). Therefore, as illustrated in FIG. 4, for example, the examiner D can insert a catheter into the urethra of the subject P lying supine on the couchtop 41 with his or her legs open and perform X-ray imaging on the urinary organs.

To conduct the urological examination described above at the end of the couchtop 41, the legs of the examiner D are placed under the couchtop, which may possibly increase the radiation exposure. For example, in the example in FIG. 4, the legs of the examiner D are placed under the couchtop. When X-rays are output from above the couchtop 41, X-rays passing through the subject P may possibly reach the legs of the examiner D.

To address this, the typical X-ray imaging apparatus 1 according to the present embodiment is provided with an X-ray protective member, such as lead acrylic resin, at the end of the couchtop 41 (reinforcement frame 421) in the longitudinal direction.

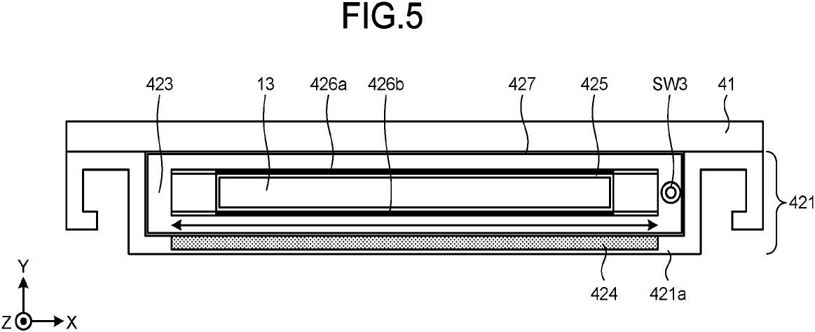

The following describes the configuration near the end of the couchtop 41 with reference to FIG. 5. FIG. 5 is a front view of the end of the couchtop 41 in the longitudinal direction according to the embodiment. In FIG. 5, the FPD tray 423 is positioned at the end of the couchtop 41 (opening 427).

As illustrated in FIG. 5, a bottom plate 421a serving as the center part of the reinforcement frame 421 is provided with an X-ray protective member 424. Specifically, the X-ray protective member 424 is provided over the area where the legs of the examiner D may possibly be placed under the couchtop. For example, in the example in FIG. 4, it is preferable to provide the X-ray protective member 424 at the protruding part of the reinforcement frame 421 under which an empty space is formed. While the X-ray protective member 424 is provided on the upper surface of the bottom plate 421a in the example illustrated in FIG. 5, the X-ray protective member 424 may be provided on the lower surface of the bottom plate 421a.

The following describes the configuration of the FPD tray 423 in detail. The FPD tray 423 includes an FPD holding part 425, guide rails 426a and 426b, and an adjustment knob SW3. The FPD holding part 425 is a holding member that insertably and removably holds the FPD 13.

The guide rails 426a and 426b are provided extending in the longitudinal direction in parallel with each other at both ends of the FPD tray 423 in the longitudinal direction. The guide rails 426a and 426b support the FPD holding part 425 with sliding members, such as sliders, interposed therebetween, thereby supporting the FPD holding part 425 slidably in the longitudinal direction of the FPD tray 423.

The FPD holding part 425 is attached to the guide rails 426a and 426b with the sliding members, such as sliders, interposed therebetween. The sliders slide on the guide rails 426a and 426b by the drive of a drive source, such as a motor and an actuator. The guide rails 426a and 426b, the sliders, and the drive source, such as a motor and an actuator, are examples of a second movement mechanism.

The adjustment knob SW3 is a knob for making fine adjustments to the position of the FPD 13. The user makes fine adjustments to the position of the FPD 13 by operating the adjustment knob SW3. The drive circuit 16 controls the FPD holding part 425 via the controller 17 and moves it in the lateral direction according to the operation of the adjustment knob SW3 by the examiner D. The fine adjustments to the position of the FPD 13 may be made using the drive switch SW1, the foot switch SW2, or other switches.

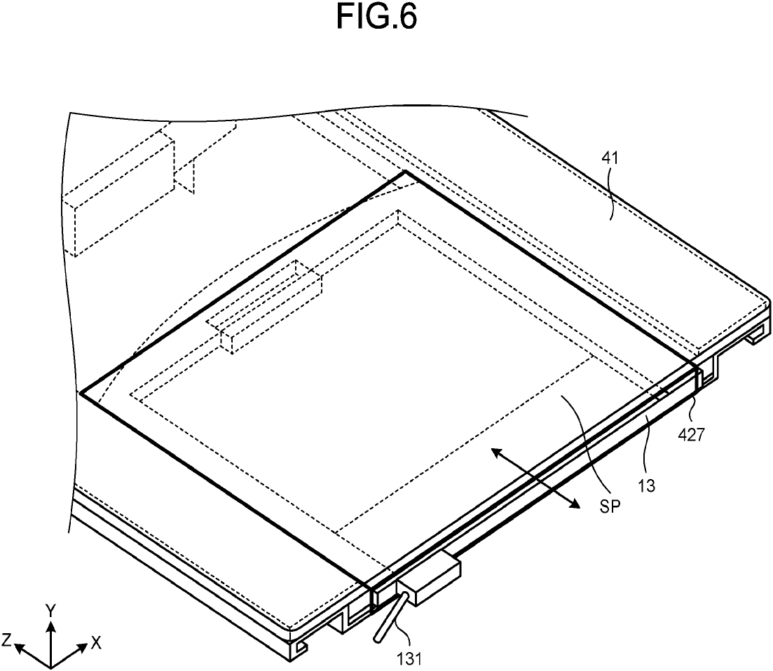

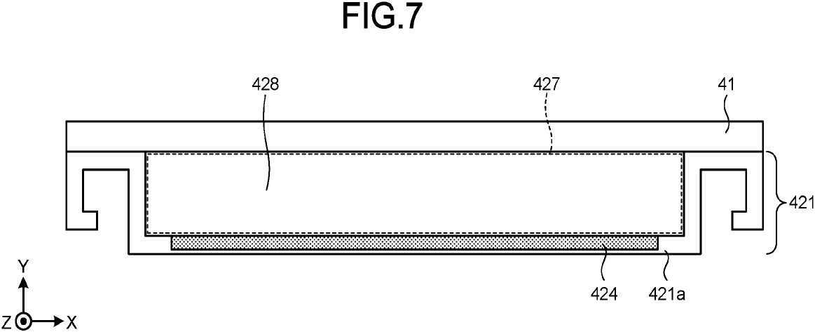

The following describes insertion and removal of the FPD 13 into and from the FPD tray 423 with reference to FIGS. 6 and 7. FIG. 6 is a view for explaining an example of insertion and removal of the X-ray detector according to the embodiment. FIG. 6 is a view of insertion and removal of the FPD 13 at the lower end of the couchtop 41 in the longitudinal direction. As illustrated in FIG. 6, the reinforcement frame 421 has the opening 427. The user inserts and removes the FPD 13 into and from the FPD tray 423 through the opening 427 as indicated by the arrow in FIG. 6.

The FPD 13 includes a locking part 131. The locking part 131 locks and prevents the FPD 13 from coming off the FPD holding part 425. To remove the FPD 13, the user unlocks the locking part 131; and when the FPD 13 is inserted, the user locks the locking part 131.

The opening 427 is provided with a shutter. The shutter is a member that opens and closes the opening 427. FIG. 7 is a view of an example of the opening when the shutter is closed according to the embodiment. FIG. 7 is a view of the opening 427 at the lower end of the couchtop 41 in the longitudinal direction. As illustrated in FIG. 7, the opening 427 is provided with a shutter 428. FIG. 7 illustrates the shutter 428 in the closed state.

When the FPD tray 423 moves to a predetermined position for inserting and removing the FPD 13, the shutter 428 is brought from the closed state into the open state. When the shutter 428 is opened, the opening 427 is in the state illustrated in FIG. 5. At this time, the shutter 428 is driven by, for example, the control of the drive circuit 16 and is housed in a space (not illustrated) between the couchtop 41 and the FPD tray 423. When the shutter 428 is opened, the user can insert and remove the FPD 13 using the opening 427.

When the FPD tray 423 moves to a position other than the predetermined position for inserting and removing the FPD 13, the shutter 428 is closed. Closing the shutter 428 can prevent dust, dirt, and other substances from entering the inside of the opening 427. Closing the shutter 428 can also prevent the user from touching the inside of the opening 427 except when necessary.

Referring back to FIG. 1, the following describes the console 20. The console 20 includes an input interface 21, a display 22, a memory 23, and circuitry 24. The console 20 according to the present embodiment is installed outside the examination room. The console 20 is not necessarily independently provided. For example, the functions of the input interface 21 and the display 22 of the console 20 may be implemented by the operation panel 33 of the imaging apparatus 10, and the functions of the memory 23 and the circuitry 24 may be implemented by the memory and the processor of the controller 17 of the imaging apparatus 10.

The input interface 21 of the console 20 is composed of, for example, typical pointing devices, such as joystick, trackball, trackball mouse, keyboard, touch panel, and numeric keypad, and hand switches for indicating an X-ray exposure timing. The input interface 21 outputs operation signals corresponding to the operations by the user to the circuitry 24.

The display 22 is composed of a typical display output apparatus, such as a liquid crystal display and an organic light-emitting diode (OLED) display and displays various kinds of information according to the control of the circuitry 24.

The memory 23 is implemented by, for example, a semiconductor memory element, such as a random access memory (RAM) and a flash memory, a hard disk, and an optical disc. For example, the memory 23 stores therein projection data.

The memory 23 stores therein dedicated computer programs for implementing an imaging control function 241, an image generation function 242, a movement control function 243, and an open/close control function 244, which will be described later.

The circuitry 24 collectively controls the operations of the typical X-ray imaging apparatus 1. The circuitry 24 includes, for example, the imaging control function 241, the image generation function 242, the movement control function 243, and the open/close control function 244.

The movement control function 243 is an example of a movement control unit.

The processing functions performed by the imaging control function 241, the image generation function 242, the movement control function 243, and the open/close control function 244 serving as the components according to the present embodiment are stored in the memory 23 in the form of computer programs executable by a computer.

The circuitry 24 is a processor that reads each computer program from the memory 23 and executes it, thereby implementing the function corresponding to the computer program. In other words, the circuitry 24 that has read the computer programs includes the functions illustrated in the circuitry 24 in FIG. 2.

In FIG. 2, the single circuitry 24 implements the processing functions performed by the imaging control function 241, the image generation function 242, the movement control function 243, and the open/close control function 244. Alternatively, the circuitry 24 may be composed of a combination of a plurality of independent processors, and the processors may implement the functions by executing the respective computer programs.

In other words, the functions described above may be provided as computer programs, and single circuitry may execute the computer programs. Alternatively, a specific function may be implemented by an independent dedicated computer program execution circuit.

The term “processor” used in the description above refers to a circuit, such as a central processing unit (CPU), a graphical processing unit (GPU), an application specific integrated circuit (ASIC), and a programmable logic device (e.g., simple programmable logic device (SPLD), complex programmable logic device (CPLD), and field programmable gate array (FPGA)).

The processor reads and executes the computer program stored in the memory 23, thereby implementing the function. Instead of being stored in the memory 23, the computer program may be embedded directly in the circuitry of the processor. In this case, the processor reads and executes the computer program embedded in the circuitry, thereby implementing the function.

The imaging control function 241 controls the X-ray tube 31, the X-ray detector 13, and the aperture unit 32 to perform X-ray imaging on the subject P. Specifically, when receiving an imaging instruction from the user, the imaging control function 241 cooperates with the movement control function 243, which will be described later, to move the center of the X-ray tube 31 to the position corresponding to an imaging region of the subject P. The imaging control function 241 controls the X-ray tube 31, the X-ray detector 13, and the aperture unit 32 and causes them to perform X-ray imaging on the subject P at the position.

To perform long imaging, the imaging control function 241 cooperates with the movement control function 243 to move the center of the X-ray tube 31 to the positions corresponding to respective imaging regions constituting the imaging range of the long image and performs X-ray imaging.

The image generation function 242 generates an X-ray image based on X-ray imaging. For example, if long imaging is performed, the image generation function 242 generates a long image based on X-ray imaging performed on a plurality of imaging regions. The X-ray image and the long image generated by the image generation function 242 are stored in the memory 23.

The movement control function 243 controls movement of each unit of the typical X-ray imaging apparatus 1. The movement control function 243 performs control for moving the X-ray tube 31 to follow the position of the X-ray detector 13 such that the positional relation between the X-ray tube 31 and the X-ray detector 13 is a predetermined positional relation.

For example, when the user operates the drive switch SW1 to move the FPD 13 (FPD tray 423) in the longitudinal direction of the couchtop 41 in the example in FIG. 3, the movement control function 243 controls the drive circuit 16 and moves the X-ray tube 31 in conjunction with the movement of the FPD 13 such that the X-ray tube 31 and the FPD 13 maintains the predetermined positional relation.

For example, when the user operates the adjustment knob SW3 to move the FPD 13 (FPD holding part 425) in the lateral direction of the couchtop 41 in the example in FIG. 5, the movement control function 243 controls the drive circuit 16 and moves the X-ray tube 31 in conjunction with the movement of the FPD 13 such that the X-ray tube 31 and the FPD 13 maintains the predetermined positional relation.

With this configuration, the movement control function 243 automatically adjusts the position of the X-ray tube 31 if the user manually makes fine adjustments to the position of the FPD 13. As a result, the user need not manually adjust the position of the X-ray tube 31 to the position of the FPD 13.

The positions of the X-ray tube 31 and the FPD 13 may be specified based on an image obtained by photographing the typical X-ray imaging apparatus 1 by the camera 34 or based on the rotation speed or the like of the motor that drives each unit of the typical X-ray imaging apparatus 1. The movement control function 243 may perform control for moving the X-ray detector 13 to follow the position of the X-ray tube 31 such that the positional relation between the X-ray tube 31 and the X-ray detector 13 is a predetermined positional relation.

The open/close control function 244 controls opening and closing of the shutter 428 that opens and closes the opening 427. For example, when the FPD tray 423 moves to the predetermined position for inserting and removing the FPD 13, the open/close control function 244 controls the drive circuit 16 and opens the shutter 428 in the closed state. For example, when the FPD tray 423 moves to a position other than the predetermined position for inserting and removing the FPD 13, the open/close control function 244 controls the drive circuit 16 and closes the shutter 428 in the open state.

With this mechanism, the user need not manually open and close the shutter 428. Therefore, the user is prevented from accidentally touching the inside of the opening 427 in situations other than inserting and removing the FPD 13. When the FPD tray 423 is moved to a position other than the position for inserting and removing the FPD 13, the open/close control function 244 performs control to close the shutter 428, thereby automatically preventing dust, dirt, and other substances from entering the inside of the opening 427.

As described above, the typical X-ray imaging apparatus 1 according to the present embodiment includes the reinforcement frame 421, the opening 427, and the FPD 13. The reinforcement frame 421 supports both ends of the couchtop 41 in the lateral direction from below and forms the space SP between the reinforcement frame 421 and the couchtop 41 in the center part corresponding to an area excluding both ends. The opening 427 is formed at an end of the couchtop 41 in the longitudinal direction and is accessible to the space SP. The FPD 13 detects X-rays and is provided in the space SP through the opening 427.

With this configuration, the typical X-ray imaging apparatus 1 according to the present embodiment moves the X-ray tube 31 to follow the position of the FPD 13, thereby enabling the region of the subject P placed at the end of the couchtop 41 in the longitudinal direction to be a target region for imaging. In other words, the typical X-ray imaging apparatus 1 according to the present embodiment can perform X-ray imaging on a region of interest of the subject P if the region of interest of the subject P is positioned at one end of the couchtop 41.

The typical X-ray imaging apparatus 1 according to the present embodiment further includes the FPD tray 423 into and from which the FPD 13 can be inserted and removed at the position of the space SP connectable to the opening 427. The FPD 13 is housed in the FPD tray 423 through the opening 427.

With this configuration, the typical X-ray imaging apparatus 1 according to the present embodiment enables the user to insert and remove the FPD 13 at the end of the couchtop 41 in the longitudinal direction. For example, if the user is a doctor who conducts a urological examination of the subject P, the typical X-ray imaging apparatus 1 facilitates the user’s inserting and removing the FPD 13 because the user is at the end of the couchtop 41 in the longitudinal direction.

The typical X-ray imaging apparatus 1 according to the present embodiment further includes a drive source, such as a motor and an actuator, that moves the FPD tray 423 in the longitudinal direction of the couchtop 41. Therefore, the user can optionally adjust the position of the FPD 13 housed in the FPD tray 423 in the longitudinal direction using the force generated by the drive source.

The typical X-ray imaging apparatus 1 according to the present embodiment further includes a drive source, such as a motor and an actuator, that moves the FPD tray 423 in the lateral direction of the couchtop 41. Therefore, the user can optionally adjust the position of the FPD 13 housed in the FPD tray 423 in the lateral direction using the force generated by the drive source.

The typical X-ray imaging apparatus 1 according to the present embodiment performs control for moving the X-ray tube 31 to follow the position of the FPD 13 such that the positional relation between the X-ray tube 31 and the FPD 13 is a predetermined positional relation.

With this configuration, the typical X-ray imaging apparatus 1 can automatically move the X-ray tube 31 such that the positional relation between the X-ray tube 31 and the FPD 13 is the predetermined positional relation. Therefore, for example, if the user manually adjusts the position of the FPD 13 to perform X-ray imaging on the subject P, the user need not manually adjust the position of the X-ray tube 31.

The typical X-ray imaging apparatus 1 according to the present embodiment further includes the X-ray protective member 424 provided under the FPD tray 423. With this configuration, the typical X-ray imaging apparatus 1 according to the present embodiment can prevent the user who performs X-ray imaging from being exposed to radiation.

The embodiment described above can be appropriately modified by changing some of the components or functions included in each apparatus. The following describes some modifications according to the embodiment described above as other embodiments. In the following description, the points different from the embodiment described above are mainly explained, and detailed explanation of the points common to the already explained contents is omitted. The modifications described below may be implemented individually or in combination as appropriate.

### First Modification

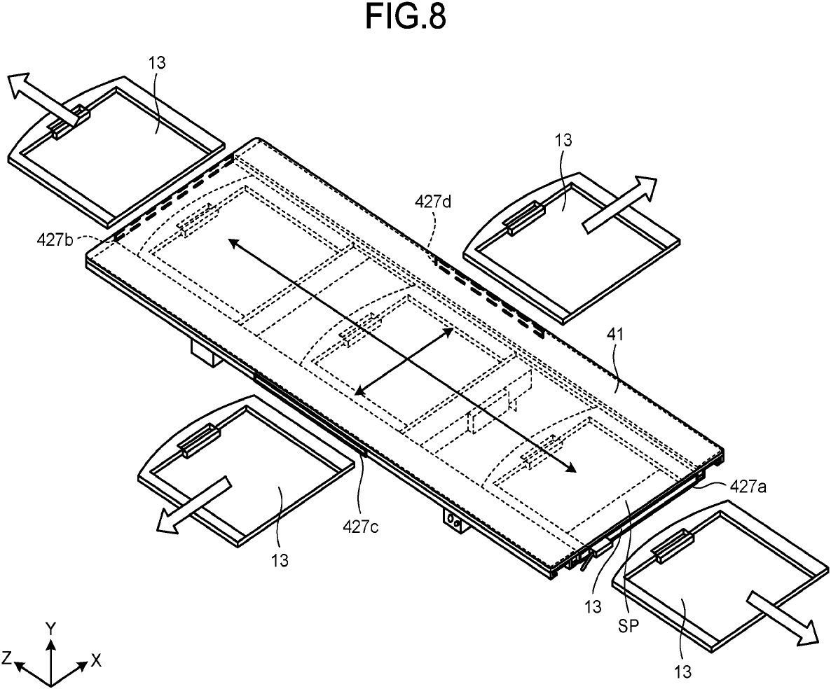

The embodiment above has described the typical X-ray imaging apparatus 1 having the opening 427 at the end of the reinforcement frame 421 in the longitudinal direction. The typical X-ray imaging apparatus 1, however, may also have openings at the ends of the reinforcement frame 421 in the lateral direction. The following describes the typical X-ray imaging apparatus 1 also having openings at the ends of the reinforcement frame 421 in the lateral direction with reference to FIG. 8.

FIG. 8 is a perspective view of an example of the configuration of the X-ray diagnostic apparatus according to a first modification. As illustrated in FIG. 8, the reinforcement frame 421 according to the present modification has a total of four openings: an opening 427a at the lower end of the couchtop 41 in the longitudinal direction, an opening 427b at the upper end in the longitudinal direction, an opening 427c at the left end of the couchtop 41 in the lateral direction, and an opening 427d at the right end in the lateral direction. In the following description, the openings 427a, 427b, 427c, and 427d may be referred to as openings 427 when they are not particularly distinguished.

The openings 427c and 427d are formed at the ends of the reinforcement frame 421 in the lateral direction, for example, by hollowing out a portion of one side surface of the reinforcement frame 421 in the longitudinal direction. As a result, the outside of the reinforcement frame 421 communicates with the space SP, whereby the openings 427c and 427d are accessible to the space SP.

Therefore, the user can insert and remove the FPD 13 using any one of the openings 427a, 427b, 427c, and 427d by aligning the position of the FPD tray 423 with the position of the openings 427a, 427b, 427c, and 427d as illustrated in FIG. 8. The FPD holding part 425 has a structure that allows insertion and removal of the FPD 13 from any direction such that the user can insert and remove the FPD 13 into and from any of the openings 427.

The present modification enables the user to change the opening 427 to be used depending on the situation, for example, when the subject P is about to come down from the couchtop 41, the user inserts and removes the FPD 13 using an opening 427 other than the one formed on the side toward which the subject P is moving. In other words, the typical X-ray imaging apparatus 1 according to the present modification can improve the convenience for the user.

### Second Modification

The first modification above has described the FPD holding part 425 having a structure that allows insertion and removal of the FPD 13 from any direction. The FPD holding part 425 may have a structure that allows insertion and removal of the FPD 13 from only one direction.

In this case, the FPD holding part 425 has a structure that allows insertion and removal of the FPD 13 on only one surface. As a result, the FPD 13 can be inserted and removed only from the opening 427 formed in the direction of the surface having the structure that allows insertion and removal of the FPD 13.

To address this, the FPD holding part 425 according to the present modification is provided to the FPD tray 423 in a manner rotatable around an axis extending in the thickness direction of the couchtop 41. The movement control function 243 according to the present modification controls the drive circuit 16 to rotate the FPD holding part 425. The movement control function 243 aligns the surface of the FPD holding part 425 having the structure that allows insertion and removal of the FPD 13 with the direction of the opening 427 to be used to insert and remove the FPD 13, thereby enabling the FPD 13 to be inserted and removed from any of the openings 427.

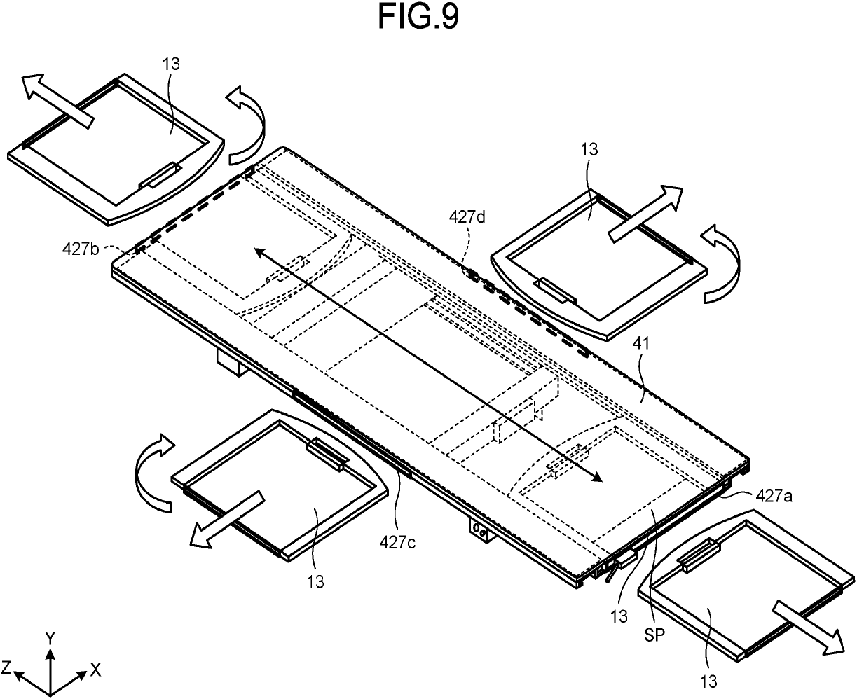

FIG. 9 is a perspective view of an example of the configuration of the X-ray diagnostic apparatus according to a second modification. For example, to insert and remove the FPD 13 from the opening 427a, the movement control function 243 rotates the FPD holding part 425 such that the surface of the FPD 13 having a rectangular shape faces the direction in which the opening 427a is formed in FIG. 9.

As a result, the surface of the FPD holding part 425 having the structure that allows insertion and removal of the FPD 13 matches the direction in which the opening 427a is formed, thereby enabling the user to insert and remove the FPD 13 from the opening 427a. The same processing described above is performed to insert and remove the FPD 13 from the other openings 427.

The present modification enables the FPD 13 to be inserted and removed from any of the openings 427 simply by providing the structure that allows insertion and removal of the FPD 13 on only one surface of the FPD holding part 425. Therefore, the present modification can prevent the structure of the FPD holding part 425 from being complicated.

### Third Modification

The embodiment above has described the X-ray protective member 424 provided outside the FPD tray 423. The X-ray protective member 424, however, may be provided inside the FPD tray 423.

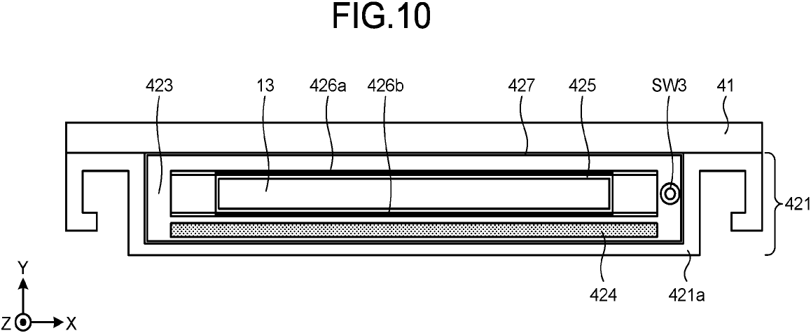

FIG. 10 is a front view of the end of the couchtop in the longitudinal direction according to a third modification. As illustrated in FIG. 10, the FPD tray 423 according to the third modification includes the X-ray protective member 424 inside thereof. The X-ray protective member 424 is provided under the FPD holding part 425. The X-ray protective member 424 according to the present modification moves with the FPD tray 423 in the long axis direction of the couchtop 41 under the control of the drive circuit 16.

The present modification can move the X-ray protective member 424 with the FPD 13. Therefore, it is unnecessary to provide the X-ray protective member 424 at both ends of the reinforcement frame 421, resulting in reduced cost.

### Fourth Modification

The embodiment above has described the couch apparatus 42 controlled by the circuitry 24 of the console 20. The couch apparatus 42, however, may have circuitry separately from the console 20. In this case, for example, the circuitry of the couch apparatus 42 controls the drive of the FPD tray 423 and the opening and closing of the shutter 428.

The present modification can distribute functions between the circuitry 24 and the circuitry of the couch apparatus 42, thereby reducing the processing load of the circuitry 24.

At least one of the embodiments described above can perform X-ray imaging at the end of the couchtop.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray diagnostic apparatus comprising:
a couchtop on which a subject is placed;
an irradiation unit configured to output an X-ray from above the couchtop;
a support member configured to support both ends of the couchtop in a lateral direction from below and form a gap between the support member and the couchtop in a center part corresponding to an area excluding both ends;
a first opening formed at either one or both of ends of the couchtop in a longitudinal direction and accessible to the gap; and
an X-ray detector provided in the gap through the first opening and configured to detect the X-ray.

2. The X-ray diagnostic apparatus according to claim 1, wherein the X-ray detector is provided in the gap in a manner insertable and removable through the first opening.

3. The X-ray diagnostic apparatus according to claim 2, further comprising:
a second opening formed at either one or both of ends of the couchtop in the lateral direction and accessible to the gap, wherein
the X-ray detector is provided in the gap in a manner insertable and removable through the first opening or the second opening.

4. The X-ray diagnostic apparatus according to claim 2, further comprising:
a housing unit into and from which the X-ray detector is capable of being inserted and removed at a position in the gap connectable to the first opening, wherein
the X-ray detector is housed in the housing unit in a manner insertable and removable through the first opening.

5. The X-ray diagnostic apparatus according to claim 4, further comprising a first movement mechanism capable of moving the housing unit in the longitudinal direction of the couchtop.

6. The X-ray diagnostic apparatus according to claim 4, further comprising a second movement mechanism capable of moving the housing unit in the lateral direction of the couchtop.

7. The X-ray diagnostic apparatus according to claim 4, further comprising:
an operating unit configured to adjust a position of the housing unit; and
a drive circuit configured to move the housing unit in the lateral direction of the couchtop according to an input to the operating unit.

8. The X-ray diagnostic apparatus according to claim 7, wherein the operating unit receives operation by hand or foot.

9. The X-ray diagnostic apparatus according to claim 5, further comprising circuitry configured to perform control for moving the irradiation unit to follow a position of the X-ray detector such that a positional relation between the irradiation unit and the X-ray detector is a predetermined positional relation.

10. The X-ray diagnostic apparatus according to claim 4, further comprising a protective member provided under the housing unit.

11. The X-ray diagnostic apparatus according to claim 10, wherein the protective member is provided inside the housing unit and under a position where the X-ray detector is housed.

12. A medical couch apparatus comprising:
a couchtop on which a subject is placed;
a support member configured to support both ends of the couchtop in a lateral direction from below and form a gap between the support member and the couchtop in a center part corresponding to an area excluding both ends; and
a first opening formed at either one or both of ends of the couchtop in a longitudinal direction and accessible to the gap.

13. The medical couch apparatus according to claim 12, wherein the first opening enables an X-ray detector configured to detect an X-ray to be inserted into or removed from the gap through the first opening.

14. The medical couch apparatus according to claim 13, further comprising:
a second opening formed at either one or both of ends of the couchtop in the lateral direction and accessible to the gap, wherein
the second opening enables the X-ray detector to be inserted into or removed from the gap through the second opening.

15. The medical couch apparatus according to claim 13, further comprising:
a housing unit into and from which the X-ray detector is capable of being inserted and removed at a position in the gap connectable to the first opening, wherein
the housing unit houses the X-ray detector in a manner insertable and removable through the first opening.

16. The medical couch apparatus according to claim 15, further comprising a first movement mechanism capable of moving the housing unit in the longitudinal direction of the couchtop.

17. The medical couch apparatus according to claim 15, further comprising a second movement mechanism capable of moving the housing unit in the lateral direction of the couchtop.

18. The medical couch apparatus according to claim 15, further comprising:
an operating unit configured to adjust a position of the housing unit; and
a drive circuit configured to move the housing unit in the lateral direction of the couchtop according to an input to the operating unit.

19. The medical couch apparatus according to claim 18, wherein the operating unit receives operation by hand or foot.

20. The medical couch apparatus according to claim 15, further comprising a protective member provided under the housing unit.
